(19) United States
(12) Patent Application Publication
(10) Pub. No.: US 2019/0160247 A1
(43) Pub. Date: May 30, 2019

(54) PROCESS AND SYSTEM FOR GENERATING PERSONALIZED FACIAL MASKS

(71) Applicant: TECHNION RESEARCH & DEVELOPMENT FOUNDATION LIMITED, Haifa (IL)

(72) Inventors: Ron KIMMEL, Haifa (IL); Nadav TOLEDO, Haifa (IL); Matan SELA, Haifa (IL); Yaron HONEN, Misgav (IL)

(21) Appl. No.: 16/313,205

(22) PCT Filed: Jun. 26, 2017
(86) PCT No.: PCT/IL2017/050705

§ 371 Date: Dec. 26, 2018

Related U.S. Application Data
(60) Provisional application No. 62/354,795, filed on Jun. 26, 2016.

(51) Int. Cl. A61M 16/06; G06F 17/50; G06T 19/20; G06T 17/00

(52) U.S. Cl. CPC A61M 16/0605; G06F 17/50; G06T 19/20; G06T 2219/2016; A61M 2016/0661; A61M 2207/00; G06T 2219/2004; G06T 17/00

(57) ABSTRACT

A process and related system and computer program product for constructing a personalized contacting interface for a facial mask, comprising the steps of: providing a three-dimensional (3D) reference model representative of a human face; identifying in the reference model a desired contact area circumscribing one or more facial regions; generating a digital design model of a contacting interface, the digital design model having a perimeter configured to provide a continuous air seal along the desired contact area of the reference model; receiving a 3D facial target model corresponding to the face of a subject; performing an elastic transformation of the reference model to conform the reference model to the target model; modifying said perimeter of said digital design model based on the deformed reference model; and using said modified digital design model to generate a set of manufacturing instructions for a contacting interface personalized for said subject.

### FIELD OF THE INVENTION

The invention relates to the field of facial m for respiratory therapy.

### BACKGROUND

Obstructive sleep apnea (OSA) is a disorder characterized by chronic pauses in breathing. Breathing is usually interrupted by a physical block of airflow caused by the soft palate, which often also leads to snoring. It can cause serious problems, including high blood pressure, mental deterioration, heart failure, sudden death, and daytime sleepiness. Surgical intervention, in which anatomical obstructions are removed, is considered in extreme cases. A more common treatment is creating an environment of continuous positive airway pressure (CPAP) to the sleeping patient. It requires the subject to wear a mask which is connected to a positive airflow generator. A CPAP mask typically comprises a mask body and a contacting interface that forms a seal around the patient's face. Ideally, the seal is air-tight under the pressure in normal service. Besides good sealing qualities, the facial mask should also feature proper fitting and comfort properties.

However, commonly-available masks are designed to fit an average face in a given population or age group. Poor or imperfect fit typically is characterized by gaps between the mask and the face, which deteriorate the impermeability of the mask and decrease the clinical effectiveness of the therapy. Trying to overcome air leak issues by fitting the mask more tightly to the patient's face can result in pressure points where the mask presses against the person's face, leading to discomfort and skin irritation. In addition, individuals have widely varying sensitivities to mechanical pressure. Discomfort and skin irritation can reduce patient tolerance and compliance with the medical procedure utilizing the mask. Therefore, a main challenge for designing a CPAP mask remains creating a mask that closely conforms to the contours of an individual's face so as to provide a consistent fit around the perimeter of the mask.

Various approaches have been attempted to address this challenge for CPAP design. These approaches include: masks with adjustable straps; masks whose overall size can be manually adjusted; masks with a cushion seal filled with a gas, liquid, or gel; masks with an inflatable cushion seal; and masks that are custom fitted for a person's face by pressing moldable material against their face.

The foregoing examples of the related art and limitations related therewith are intended to be illustrative and not exclusive. Other limitations of the related art will become apparent to those of skill in the art upon a reading of the specification and a study of the figures.

### SUMMARY

The present invention provides a process, and related system and computer program product, for constructing a personalized contacting interface for a facial mask.

According to a first aspect, the process for constructing a personalized contacting interface for a facial mask comprises the steps of: providing a three-dimensional (3D) reference model representative of a human face; identifying in the reference model a desired contact area circumscribing one or more facial regions; generating a digital design model of a contacting interface, the digital design model having a perimeter configured to provide a continuous air seal along the desired contact area of the reference model; receiving a 3D facial target model corresponding to the face of a subject; performing an elastic transformation of the reference model to conform the reference model to the target model; modifying said perimeter of said digital design model based on the deformed reference model; and using said modified digital design model to generate a set of manufacturing instructions for a contacting interface personalized for said subject.

According to another aspect, there is provided a system for constructing a personalized contacting interface for a facial mask, the system comprising: at least one hardware processor; and a processor-attached non-transitory computer-readable storage medium having program code embodied therewith, the program code executable by the at least one hardware processor to: provide a three-dimensional (3D) reference model representative of a human face; identify in the reference model a desired contact area circumscribing one or more facial regions; generate a digital design model of a contacting interface, the digital design model having a perimeter configured to provide a continuous air seal along the desired contact area of the reference model; receive a 3D facial target model corresponding to the face of a subject; align the reference model with the target model; perform an elastic transformation of the reference model to conform the reference model to the target model; modify said perimeter of said digital design model based on the deformed reference model; and use said modified digital design model to generate a set of manufacturing instructions for a contacting interface personalized for said subject.

According to another aspect, there is provided a computer program product for constructing a personalized contacting interface for a facial mask, the computer program product comprising a non-transitory computer-readable storage medium having program code embodied therewith, the program code executable by at least one hardware processor to provide a three-dimensional (3D) reference model representative of a human face; identify in the reference model a desired contact area circumscribing one or more facial regions; generate a digital design model of a contacting interface, the digital design model having a perimeter configured to provide a continuous air seal along the desired contact area of the reference model; receive a 3D facial target model corresponding to the face of a subject; align the reference model with the target model; perform an elastic transformation of the reference model to conform the reference model to the target model; modify said perimeter of said digital design model based on the deformed reference model; and use said modified digital design model to generate a set of manufacturing instructions for a contacting interface personalized for said subject.

In some embodiments, the reference model comprises at least the nasal region and oral region of a human face. In some embodiments, the reference model is provided in a format selected from the group consisting of polygon mesh, depth map, parameterized polynomial, and subspace representation. In some embodiments, the step of providing a reference model further comprises the step of selecting from among a plurality of provided 3D model representative of various face shapes.

In some embodiments, said desired contact area circumscribes at least one of the nasal region and the oral region of a face. In one embodiment, said desired contact area circumscribes the entire face.

In some embodiments, said facial mask comprises a standard mask body, wherein said contacting interface is interchangeable and is configured to be associated with said standard mask body. In some embodiments, said facial mask is a continuous positive airway pressure (CPAP) mask.

In some embodiments, generating said digital design model of a contacting interface comprises generating a plurality of digital design models associated with various face shapes. In some embodiments, generating said digital design model of a contacting interface comprises the steps of: receiving a digital design model of a contacting interface, and modifying said perimeter of said received digital design model so as to provide a continuous air seal along said desired contact area of the reference model.

In some embodiments, performing an elastic transformation of the reference model comprises an initial step of performing a rigid alignment of the reference model with the target model. In some embodiments, said rigid alignment comprises the steps of: identifying a plurality of first feature points (FPD) corresponding to selected facial locations of the reference model; detecting a plurality of second FPDs of the target model corresponding to said plurality of first FPDs; and finding a transformation correlating said plurality of first FPDs with said plurality of second FPDs, such that the geometric distance between the two sets of FPDs is minimized. In some embodiments, said plurality of first FPDs comprises at least a nose tip, mouth corners, and eye corners. In some embodiments, said plurality of second FPDs is detected automatically. In some embodiments, performing an elastic transformation of the reference model comprises employing an iterative closest point process.

In some embodiments, modifying said perimeter of said digital design model comprises the steps of: identifying a plurality of first control points along said desired contact area of the reference model; identifying a plurality of second control points along said perimeter of said digital design model, corresponding to said plurality of first control points; and modifying the position of said plurality of second control points based upon the shifted coordinates of said plurality of first control points.

In some embodiments, generating a set of manufacturing instructions generates a set of instructions to construct a mold. In some embodiments, the step of generating a set of manufacturing instructions generates a set of instructions to manufacture said contacting interface in an additive printing process.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1A illustrates the main parts of a generic CPAP mask.

FIG. 1B is a schematic illustration of a mask system comprising an interchangeable contacting interface.

FIG. 2 illustrates a block diagram of an embodiment of the process for generating personalized facial masks, according to embodiments of the present invention.

FIG. 3 illustrates a 3D model of a generic human face, according to embodiments of the present invention.

FIG. 4 illustrates a digital design model of a contacting interface, according to embodiments of the present invention.

FIG. 5 illustrates a 3D scan of the face of a subject in various stages of processing, according to embodiments of the present invention.

FIG. 6 illustrates a generic reference model having been conformed to the shape of the face of a subject, according to embodiments of the present invention.

FIG. 7 illustrates the results of a test to evaluate the fit of the personalized facial mask, according to embodiments of the present invention.

### DETAILED DESCRIPTION

Disclosed herein is a process and system for designing and optionally also manufacturing a personalized facial mask, of the type that requires a tight seal around one or more facial features—such as the nose, mouth, and eyes.

A prominent example of a mask compatible with the present invention is a CPAP mask, but the invention is certainly not limited to this particular mask type. Some embodiments, therefore, pertain to a CPAP mask interface for patients suffering from OSA. The disclosed process is quick and efficient, and does not require manual input or intervention.

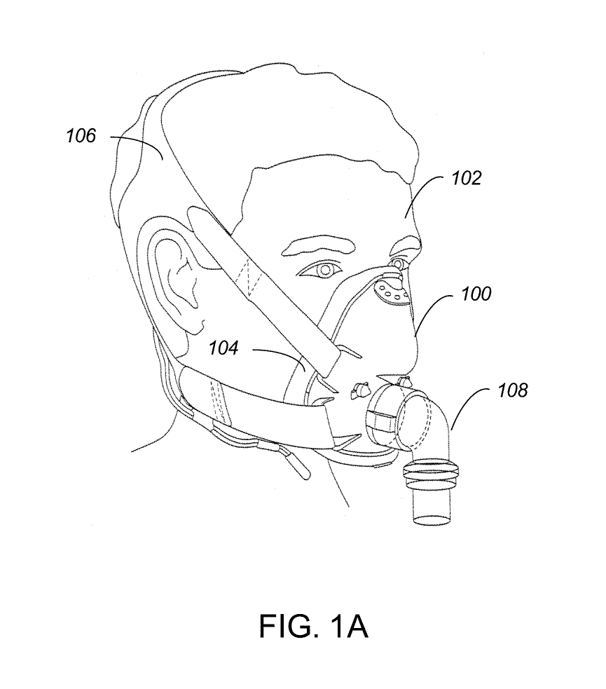

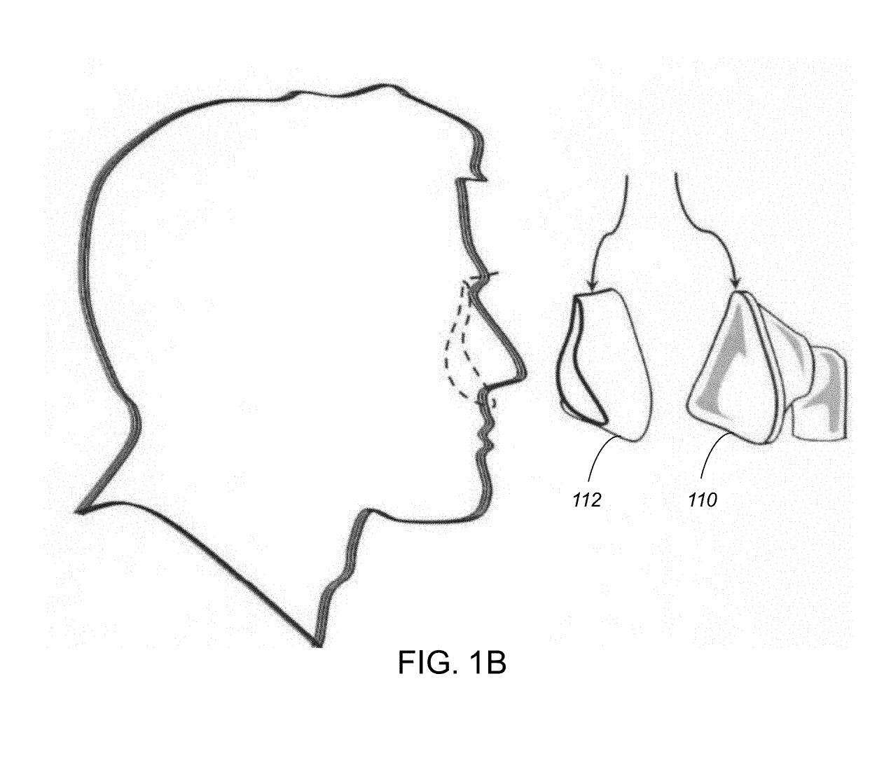

FIG. 1A shows an illustration of a common respiratory-assisting mask in order to provide context for introducing the present process. There are shown, in relevant parts, a main body 100 of the mask that forms a compartment containing breathable gas, which covers the person's nose and mouth; a face-contacting interface 104 of the mask that forms a seal between mask 100 and the person's face 102; straps 106 that attach the main body of the mask 100 to the person's head; and breathable gas tube 108.

Respiratory masks may take various forms. In some variations, a face-contacting perimeter interface, such as interface 104, may be constructed to be associated with a standard mask body 100 as an interchangeable element. In other variations, mask body 100 and interface 104 may comprise a single element configured to be associated with straps 106 and tube 108. In this example, the respiratory-assisting mask covers both the person's nose and mouth. In other examples, the respiratory-assisting mask may cover only the person's nose or only the person's mouth. In yet other examples, the mask may cover a person's entire face. In addition, similar masks may employ various methods by which the mask is attached to the person's head. A clearer view of a contacting interface is provided in FIG. 1B, wherein interface 110 is configured as an interchangeable element of respiratory mask system 110, which is a nasal mask, in this case.

By way of overview, the present process begins by providing a three-dimensional (3D) model of a generic human face, which will be termed the “reference model” within the present process. On the reference model is identified a desired contact area for a mask interface. Such contact area typically includes the perimeter of the nasal and/or oral regions of the face. A digital design model for a mask interface fitting the identified contact area is then generated. Next, a 3D scan of the face of a patient is obtained; such 3D scan will be known as the “target model” within the present process. The process then aligns (or “registers”) the generic model with the target model, and performs an elastic transformation procedure whereby the reference model is conformed to the surface of the target model. In the course of the elastic transformation, the contact region previously identified on the reference model is warped to acquire the precise contours of the corresponding area of face of the subject. The shifted coordinates of the warped contact area can then be applied to personalize the digital design model with respect to the specific subject. The resulting personalized digital design model can then be produced, for example, via additive printing technology, by directly printing the model, or by printing a mold thereof. An evaluation of the efficiency of the present process was performed by estimating the force variations along the contact region between the mask interface and the face of a subject. It was found that the present process offered improvements over currently available designs. Specifically, it was found that the present process provided for uniformly distributed pressure along the contact area between the mask interface and the face.

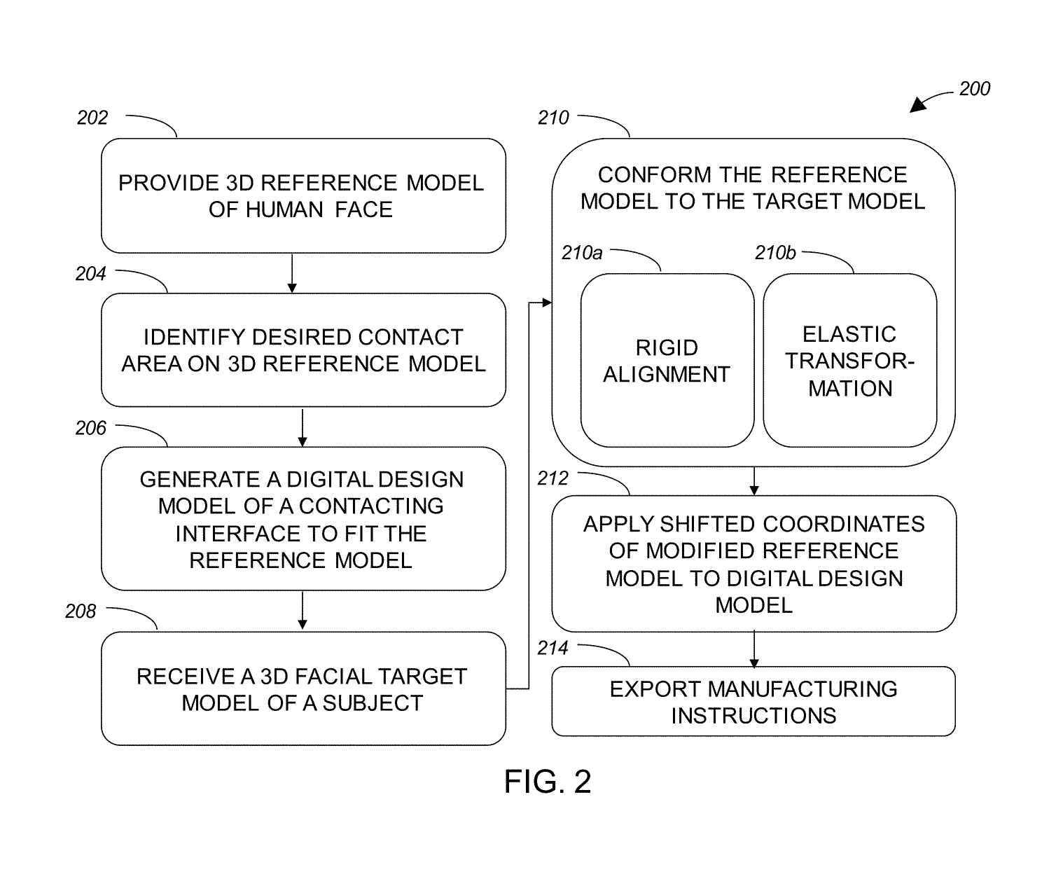

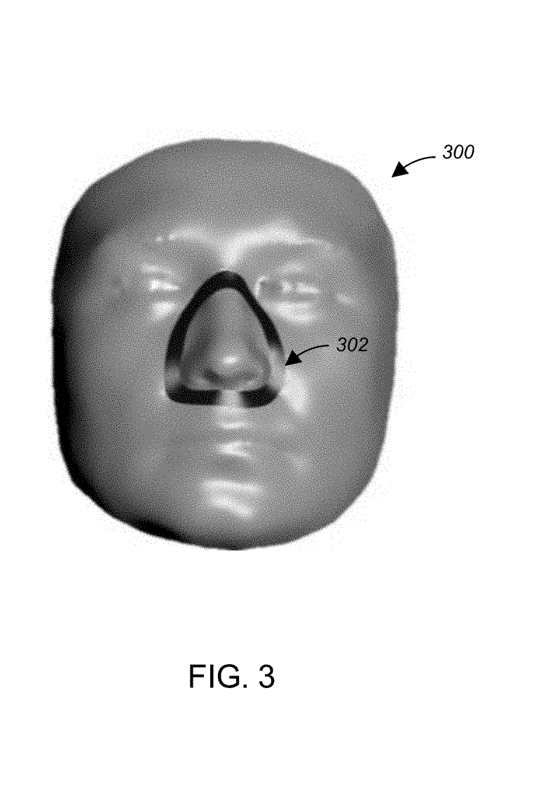

The particulars of the present process will now be described with reference to the drawings. FIG. 2 illustrates a flowchart of an exemplary embodiment of a personalized facial mask generation process 200. In a first step 202, a reference model comprising a 3D representation of a generic human face is provided. For exemplary purposes, the process of step 202 will be described herein with reference to the components of a reference model, shown rendered at 300 in FIG. 3. The term “generic human face” as used herein is a broad term and includes, without limitation, any 3D representation of a human face comprising the anatomical regions of a human face or relevant parts thereof, and generally having non-prominent facial features. In some variations, the reference model 300 may be generated from a model rendered by an artist. In other variations, the reference model 300 represents an average composite human face computed from a plurality of known faces. The reference model 300 may further be provided in various configurations and formats, including as a polygon mesh, a depth map, a parameterized polynomial, or a subspace representation. In certain variations, the reference model 300 comprises a plurality of landmark points that indicate the likely location and size of facial features (e.g., eyes, nose, mouth, ears). In still other variations, there may be provided more than one reference model, representing a variety of facial types, shapes, and sizes.

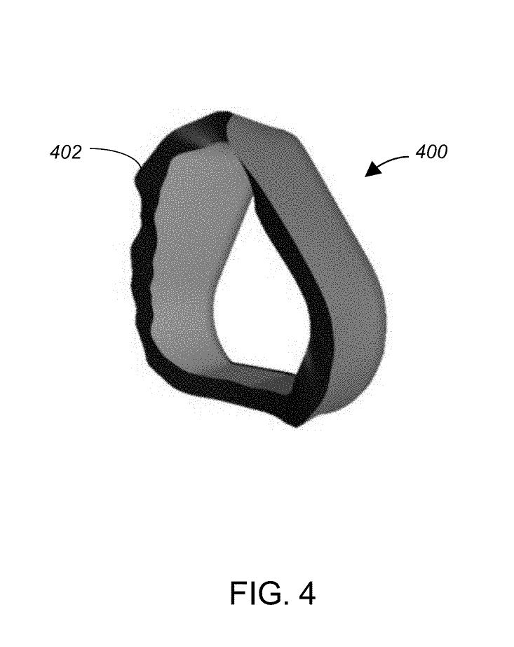

With continued reference to FIG. 2, in a step 204, there is identified within the reference model 300 of FIG. 3 a contact area 302. The contact area 302 generally circumscribes a desired facial region which may comprise the nasal area and/or the oral area of the human face. The contact area 302 delineates the contours along which a contacting interface will touch the surface of the face. In a following step 206, a digital design model of a contacting interface is generated using a computer-aided design (CAD) tool, the contacting interface model being configured to provide an air seal along the contact area 302. FIG. 4 illustrates an example of such a digital design model of a contacting interface 400, having a face-contacting perimeter 402. In a variation, a suitable design model of a contacting interface may be received as a CAD file, whereby step 206 may comprise modifying points comprising the perimeter 402 so as to fit the contact area 302. It will be appreciated that the process steps 204 and 206 are preparatory set-up steps, which need only be performed once with respect to each type of a contacting interface desired to be generated in accordance with the present process.

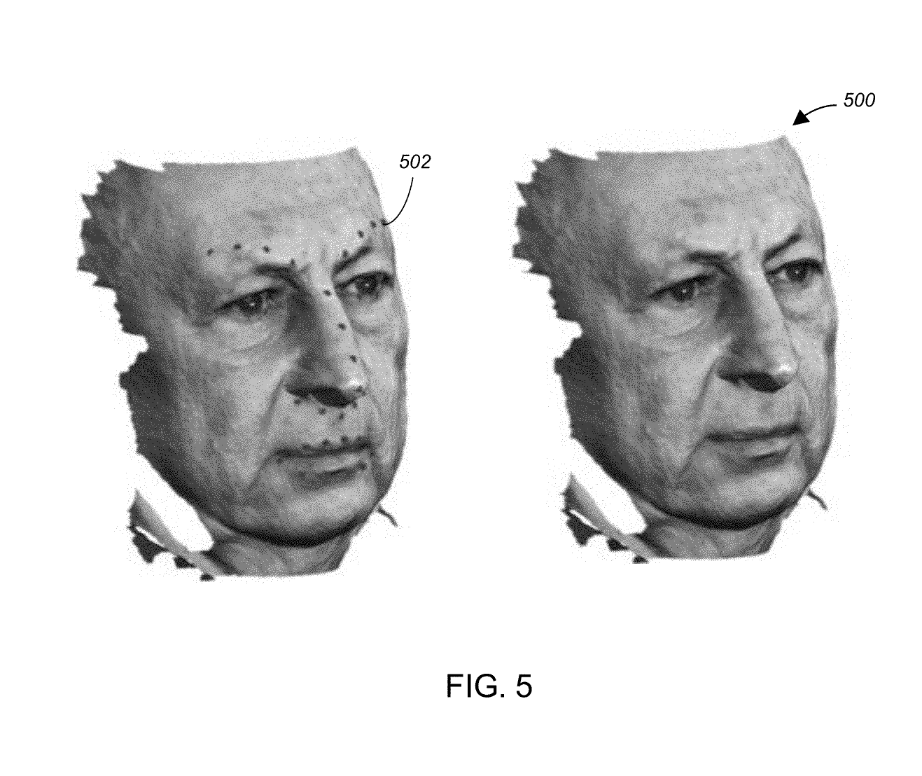

In a next step 208 of FIG. 2, a 3D scan of the face of a subject is received (or is actively performed as part of the method, using a suitable 3D scanner or 3D imaging apparatus) and designated as the “target model” within the process 200. The target model may be generated using any commercially available 3D imaging technique, and may be provided in various configurations and formats, including as a polygon mesh, a depth map, a parameterized polynomial, or a subspace representation. An example of a target model 500 is provided in FIG. 5. In contrast to the generic reference model 300 of FIG. 3, the target model 500 represents a faithful reproduction of a particular human face. It will be appreciated that step 208 may comprise the sub-steps of (i) selecting from among a plurality of captured 3D images based upon a qualitative score assigned to each image, (ii) synthesizing a plurality of full and/or partial individual images of the face of the subject into the target model, and (iii) applying a compression and/or sub-sampling process in order to decrease the amount of data captured in the target model.

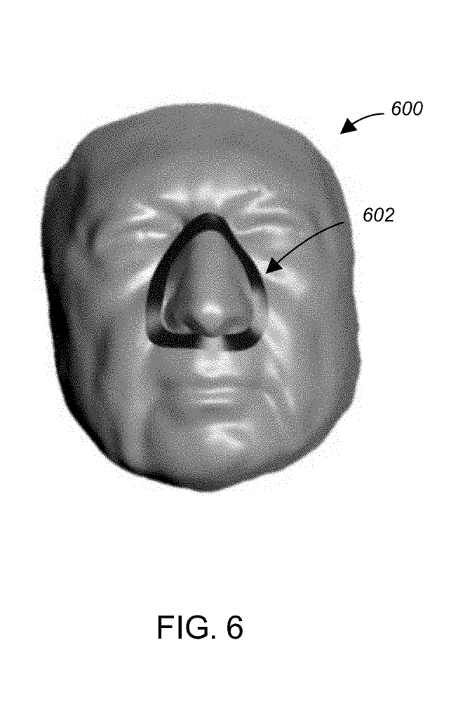

In a next step 210 of FIG. 2, there is performed an image transformation process which conforms the reference model to the target model. More specifically, an automatic deformation technique is used to align the features of the reference model with the corresponding features of target model. The deformation procedure of step 210 results in a modified reference model, which faithfully reflects the geometric features of the specific subject. An example of such modified reference model is provided in a reference model 600 of FIG. 6. It will be appreciated that, in the course of this process, the pre-determined “generic” contact area 302 of FIG. 3 is transformed into a “personalized” contact area 602 of FIG. 6, which now conforms precisely to the contours of the respective area of the face of the subject.

Following is a discussion of the particulars of the transformation process of step 210. The transformation process step 210 may advantageously comprise an initial alignment in a sub-step 210a, whereby the reference mage is transformed rigidly, (i.e., as an entire image, without local deformation) within the coordinate system to be brought into feature-based alignment with the target model. In order to perform this initial alignment, in one variation, a plurality of first salient facial landmarks is identified in the reference model, as a preparatory step. These landmarks, or feature points (FPD), can include, but are not limited to, points on the chin, nostrils, peripheral regions of the eye lids, eyebrows, lips and mouth, combinations of the same, or the like. In certain variations, the FPDs advantageously include at least points corresponding to the nose tip, eye corners, and mouth corners. Then, a corresponding plurality of second FPDs is detected in the target model. Advantageously, the FPDs of the target model are detected automatically using any method of facial landmark detection of digital face data, such as an active shape model. FIG. 5 illustrates an exemplary head model with identified FPDs (such as FPD 502) corresponding generally to characteristic points or regions on an individual's face, in accordance with certain variations of the invention. In practice, it is estimates that approximately 60 FPDs are used, however, more or fewer FPDs can be used. There is then employed in sub-step 210a an iterative algorithm configured to find a transformation mapping the plurality of first FPDs to the plurality of second FPDs, such that the geometric distance between the two sets of FPDs is minimized. More specifically, denoting the plurality of first FPDs as {r₁^{temp}, . . . , rⱼ^{temp}} and the plurality of second FPDs as {r₁^{scan}, . . . , rⱼ^{scan}}, the iterative algorithm of sub-step 210a first calculates the scaling factor α by minimizing the term:

$${min}_{α} {\sum }_{i , j}^{k} {‖ α \cdot d ( {r}_{i}^{temp} , {r}_{j}^{temp} ) - d ( {r}_{i}^{scan} , {r}_{j}^{scan} ) ‖}_{2}^{2} .$$

where, d( . . . ) represents the Euclidean distance between each pair of FPDs.

Next, the algorithm of step 210a calculates the rotation matrix R, the translation vector t, and updates the scaling factor α, iteratively, by minimizing the term:

$${min}_{R ∈ so ( 3 ) , t ∈ {R}^{3} , α ∈ {R}_{+}} {\sum }_{i = 1}^{k} {‖ ( α {Rr}_{i}^{temp} + t ) - {r}_{i}^{scan} ‖}^{2}$$

This process converges after several iterations with an accurate rigid transformation of the reference model.

A subsequent, elastic, transformation is then performed in a sub-step 210b to locally deform the reference model to conform to the precise geometry of the target model. In certain variations, the elastic transformation process may be performed in a single step. In certain variations, there may employed an iterated closest point (ICP) algorithm or process. Such an iterative process can include, for example, the following steps:

First, the algorithm associates a plurality of surface points of the reference model and target model using nearest neighbor criteria. A K-dimensional tree is constructed comprising the surface points of the target model. The nearest neighbor algorithm then finds, for each point of the reference model vᵢ^{temp}, a close point on the target model cᵢ^{scan}.

Second, the algorithm removes from the obtained list outliers, which may be the result of holes and/or noise in the target image. Such outliers may be defined for this purpose as matching pairs (i) which are more than five millimeters apart, or (ii) whose normal directions differ at more than twenty-five degrees.

Third, the algorithm performs an elastic deformation of the reference model using the remaining matching pairs. The deformation is modeled as an optimization of the change in position of each surface point of the reference model within a displacement field d=(d₁^{temp}, . . . , dₙ_{ₜₑₘₚ}^{temp}), such that:

E(d)=αₚ₂ₚₒᵢₙₜ·Eₚ₂ₚₒᵢₙₜ(d)+αₚ₂ₚₗₐₙₑ·Eₚ₂ₚₗₐₙₑ(d)+α_{memb}·E_{memb}(d)+α_{ref}·E_{re f}(d),

where α(.) represent positive scalar weights, and the energy terms are given by:
Point-to-point energy: The sum of squared Euclidean distances between corresponding points vᵢ^{temp} of the reference model and cᵢ^{scan} of the target model:

$${E}_{p 2 point} ( d ) = {\sum }_{i = 1}^{p ′} {‖ {v}_{i}^{temp} + {d}_{i}^{temp} - {c}_{i}^{scan} ‖}^{2} .$$

Point-to-plane energy: The sum of squared Euclidean distances between a point vᵢ^{temp} of the reference model and the tangent plane of the corresponding point cᵢ^{scan} of the target model:

$${E}_{p 2 plane} ( d ) = {\sum }_{i = 1}^{p ′} {\vert {n}_{i}^{scan} \cdot {( {v}_{i}^{temp} + {d}_{i}^{temp} - {c}_{i}^{scan} )}^{T} \vert}^{2} ,$$

where nᵢ^{scan} is the unit normal at the point cᵢ^{scan}.
Biharmonic energy: This regularization term enforces the smoothness of the displacement field as functions on the reference model:

$${E}_{memb} ( d ) = {\sum }_{i = 1}^{{n}_{temp}} {\sum }_{j ∈ 𝒩 ( {v}_{i}^{temp} )} {‖ {w}_{i , j} ( {d}_{i}^{temp} - {d}_{j}^{temp} ) ‖}^{2} ,$$

where w_{i,j} are the cotangent weights and [symbol] (vᵢ^{temp}) are the set of neighboring vertices of vᵢ^{temp}.
Constraint energy: This term measures the sum of squared Euclidean distances between the detected corresponding feature points:

$${E}_{ref} ( d ) = {\sum }_{i = 1}^{k} {‖ {r}_{i}^{temp} + {d}_{i}^{temp} - {r}_{i}^{scan} ‖}^{2} .$$

Fourth, the previous step is repeated gradually in a coarse-to-fine fashion by adjusting the relative scalar weights α(.). Initially, the relative scalar weights are set as αₚ₂ₚₒᵢₙₜ=0.1, αₚ₂ₚₗₐₙₑ=1, α_{memb}=100, and α_{ref}=10. With each new iteration, the norm of the displacement field is measured relative to the previous iteration. If the value is below 10⁻², the α_{memb} and α_{ref} weightings are decreased by half. This algorithm converges typically after 10 to 20 iterations with an accurate and smooth alignment.

At the conclusion of this sub-step 210b, a plurality of individual surface points defining the three-dimensional geometry of the reference model is shifted within the 3D coordinate system based upon the location of a plurality of corresponding surface points of the target model.

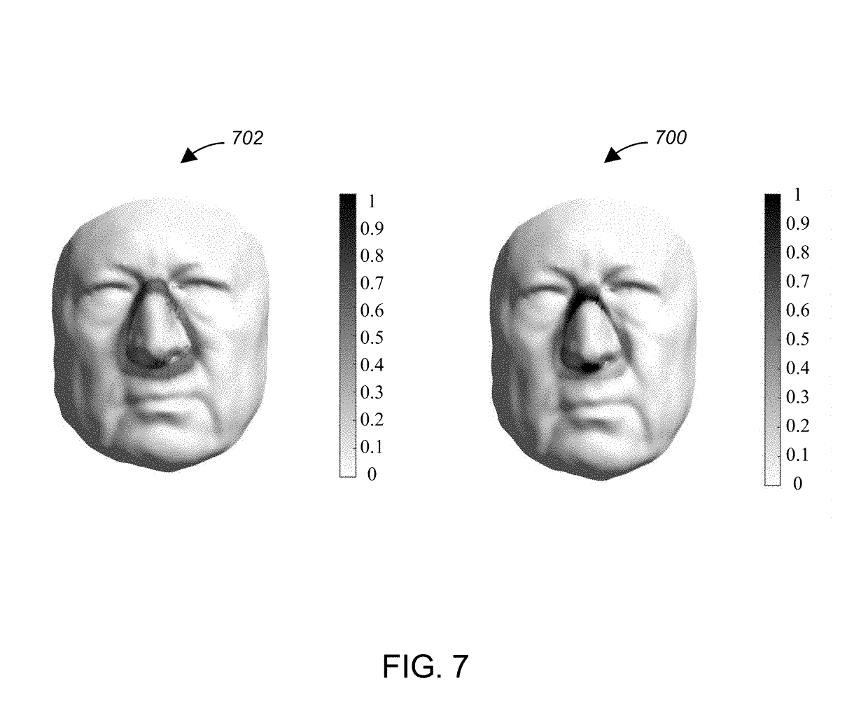

A subsequent step 212 provides for the process of applying the shifted coordinates of the deformed reference model, and, specifically, the shifted coordinates of the “personalized” contact area 602 of FIG. 6, to the digital design model 400 of FIG. 4, such that the perimeter 402 is transformed to fit the deformed reference model, and by extension, the facial contours of the subject. For that purpose, the perimeter 402 of the digital model 400 may, for example, be assigned a plurality of control points evenly spread about its surface. By editing the position of said control points based on the modified positions of corresponding control points of the “personalized” contact are 602, the perimeter 402 may be transformed as desired. In practice, 256 such control points may be used, however, more or fewer control points may be used. The digital design model so modified may then be exported in a step 214 of FIG. 2 as a set of manufacturing instructions, e.g., for (i) producing a mold of the contacting interface into which is then injected a suitable material for producing the final product, or (ii) printing directly a contacting interface using an additive printing process. FIG. 7 illustrates the results of an experiment to evaluate the effectiveness of the personalized facial mask, according to embodiments of the present invention. The experiment was conducted by comparing the force variations along the contact region between the contacting interface and the face of a subject. It was found that, as compared with a currently available design (in a simulation 700), a personalized contacting interface produced in accordance with embodiments of this invention provided for a more uniformly distributed pressure along the contact area between the mask interface and the face (in a simulation 702).

As will be appreciated by one skilled in the art, aspects of the present invention may be embodied as a system, process or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The system disclosed in the present specification may further be specially constructed for the required purposes, or may comprise a general purpose computer or other device selectively activated or reconfigured by a computer program stored in the computer. The algorithms presented herein are not inherently related to any particular computer or other apparatus. Various general purpose machines may be used with programs in accordance with the teachings herein. Alternatively, the construction of more specialized system to perform the required method steps may be appropriate.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A process for constructing a personalized contacting interface for a facial mask, comprising the steps of:
providing a three-dimensional (3D) reference model representative of a human face;
identifying in the reference model a desired contact area circumscribing one or more facial regions;
generating a digital design model of a contacting interface, the digital design model having a perimeter configured to provide a continuous air seal along the desired contact area of the reference model;
receiving a 3D facial target model corresponding to the face of a subject;
performing an elastic transformation of the reference model to conform the reference model to the target model;
modifying said perimeter of said digital design model based on the transformed reference model; and
using said modified digital design model to generate a set of manufacturing instructions for a contacting interface personalized for said subject.

2. The process of claim 1, wherein the reference model comprises at least the nasal region and oral region of a human face.

3. The process of claim 1, wherein the reference model is provided in a format selected from the group consisting of polygon mesh, depth map, parameterized polynomial, and subspace representation.

4. The process of claim 1, wherein the step of providing a reference model further comprises the step of selecting from among a plurality of provided 3D model representative of various face shapes.

5. The process of claim 1, wherein said desired contact area circumscribes at least one of the nasal region and the oral region of a face.

6. The process of claim 1, wherein said desired contact area circumscribes the entire face.

7. The process of claim 1, wherein said facial mask comprises a standard mask body, and wherein said contacting interface is interchangeable and is configured to be associated with said standard mask body.

8. The process of claim 1, wherein said facial mask is a continuous positive airway pressure (CPAP) mask.

9. The process of claim 1, wherein the step of generating said digital design model of a contacting interface comprises generating a plurality of digital design models associated with various face shapes.

10. The process of claim 1, wherein the step of generating said digital design model of a contacting interface comprises the steps of:
receiving a digital design model of a contacting interface, and
modifying said perimeter of said received digital design model so as to provide a continuous air seal along said desired contact area of the reference model.

11. The process of claim 1, wherein the step of performing the elastic transformation of the reference model comprises an initial step of performing a rigid alignment of the reference model with the target model.

12. The process of claim 11, wherein said rigid alignment comprises the steps of:
identifying a set of first feature points (FPD) corresponding to selected facial locations of the reference model;
detecting a set of second FPDs of the target model corresponding to said plurality of first FPDs; and
finding a transformation which minimizes a geometric distance between said set of first FPDs and said set of second FPDs.

13. The process of claim 12, wherein said set of first FPDs comprises at least a nose tip, mouth corners, and eye corners.

14. The process of claim 12, wherein said set of second FPDs is detected automatically.

15. The process of claim 1, wherein the step of performing the elastic transformation of the reference model comprises employing an iterative closest point process.

16. The process of claim 1, wherein the step of modifying said perimeter of said digital design model comprises the steps of:
identifying a plurality of first control points along said desired contact area of the transformed reference model;
identifying a plurality of second control points along said perimeter of said digital design model, corresponding to said plurality of first control points; and
modifying a position of each of said plurality of second control points based upon a position of each of said plurality of first control points.

17. The process of claim 1, wherein the step of generating a set of manufacturing instructions generates a set of instructions to construct a mold.

18. The process of claim 1, wherein the step of generating a set of manufacturing instructions generates a set of instructions to manufacture said contacting interface in an additive printing process.

19. A system comprising:
at least one hardware processor; and
a processor-attached non-transitory computer-readable storage medium having program code embodied therewith, the program code executable by the at least one hardware processor to:
provide a three-dimensional (3D) reference model representative of a human face,
identify in the reference model a desired contact area circumscribing one or more facial regions,
generate a digital design model of a contacting interface, the digital design model having a perimeter configured to provide a continuous air seal along the desired contact area of the reference model,
receive a 3D facial target model corresponding to the face of a subject,
align the reference model with the target model,
perform an elastic transformation of the reference model to conform the reference model to the target model,
modify said perimeter of said digital design model based on the transformed reference model, and
use said modified digital design model to generate a set of manufacturing instructions for a contacting interface personalized for said subject.

20. The system of claim 19, wherein the reference model comprises at least the nasal region and oral region of a human face.

21-54. (canceled)
